(19) United States
(12) Patent Application Publication
(10) Pub. No.: US 2002/0094519 A1
(43) Pub. Date: Jul. 18, 2002

(54) Detection of protein interactions

(75) Inventors: Kevin J. McKernan, Marblehead, MA (US); Joel A. Malek, Beverly, MA (US); Paul J. McEwan, Cambridge, MA (US)

Correspondence Address: LOUIS MYERS, Fish & Richardson P.C., 225 Franklin Street, Boston, MA, 02110-2804, US

(73) Assignee: AGENCOURT BIOSCIENCE CORPORATION

(21) Appl. No.: 10/092,825

(22) Filed: Mar. 7, 2002

(51) Int. Cl. C12Q 1/00; C12Q 1/68

(52) U.S. Cl. 435/4; 435/6

(57) ABSTRACT

The present invention provides methods for determining the sequence of nucleic acids encoding interacting polypeptide sequences. Two hybrid assays are carried out to select host cells containing nucleotide sequences encoding interacting proteins. The identity of the nucleotide sequences is determined by isolating the nucleotide sequences from the selected host cells and carrying out sequencing reactions on the nucleotide sequences.

### FIELD OF THE INVENTION

The invention relates to compositions and methods for the identification of nucleic acid sequences encoding interacting proteins.

### BACKGROUND OF THE INVENTION

A variety of assay systems are available for detecting interactions between two proteins. Examples of such assay systems include the co-precipitation of bound polypeptides as well as two hybrid assays.

Interactions between two proteins can be been detected by performing co-precipitation experiments in which an antibody to a known protein is mixed with a cell extract and used to precipitate the known protein and any proteins that are stably associated with it. According to such methods, the nucleotide sequences encoding interacting proteins can be determined by first determining the amino acid sequence of a polypeptide associated with the known protein, and using that amino acid sequence information to determine the nucleotide sequence encoding the bound polypeptide. The nucleotide sequence can be determined either by cloning methodologies or by comparison to sequences contained in electronic databases.

In two hybrid assays (also referred to as interaction trap systems) polypeptide sequences are identified that bind to a predetermined, known polypeptide sequence present in a fusion protein (Fields and Song (1989) Nature 340:245). The two hybrid methodology can identify protein-protein interactions in a cell through reconstitution of a transcriptional activator. In general, two hybrid assays are carried out to select cells wherein a bait protein binds to a prey protein. Two hybrid assays typically use a known protein as a bait to identify an unknown protein (prey) or proteins that bind to the bait protein. The application of two hybrid methods in bacterial systems has been described by, e.g., Dove et al. (1998) Genes & Development 12:745-54 and U.S. Pat. No. 5,925,523.

### SUMMARY OF THE INVENTION

The invention is based, at least in part, on the discovery that nucleotide sequences can be selected for sequencing based upon their ability to encode interacting bait and prey fusion proteins in a two hybrid assay system. In the methods described herein, nucleotide sequences are used to encode bait and prey fusion partners, without necessarily determining beforehand the identity of either of the nucleotide sequences used in the two hybrid assay. Accordingly, the detection of an interaction between a bait and a prey fusion protein in a host cell is used as an indicator that the host cell contains nucleotide sequences encoding interacting polypeptides. Upon the detection of such an interaction, the nucleotide sequences are then sequenced to reveal their identity.

In one aspect, the invention features a method for identifying nucleotide sequences encoding interacting polypeptide sequences, wherein the method includes the steps of: (1) providing a host cell containing a reporter gene operably linked to a transcriptional regulatory sequence which includes a binding site for a DNA-binding domain; (2) introducing into the host cell a first chimeric gene encoding a first fusion protein, wherein the first fusion protein contains a first polypeptide sequence and a DNA binding domain, and wherein the first chimeric gene contains a first nucleotide sequence encoding the first polypeptide sequence; (3) introducing into the host cell a second chimeric gene encoding a second fusion protein, wherein the second fusion protein contains a second polypeptide sequence and an activation tag, and wherein the second chimeric gene contains a second nucleotide sequence encoding the second polypeptide sequence; (4) culturing the host cell for a time sufficient to allow an interaction of the first fusion protein and the second fusion protein, wherein the interaction results in a measurable change in expression of the reporter gene; (5) selecting the host cell based upon the measurable change in expression of the reporter gene; and (6) sequencing the first nucleotide sequence and the second nucleotide sequence, to thereby identify nucleotide sequences encoding interacting polypeptide sequences.

The host cell can be a prokaryotic cell (e.g., a bacterial cell such as Escherichia coli) or a eukaryotic cell (e.g., a yeast cell or a mammalian cell).

The first and/or second nucleotide sequence can be derived from a nucleic acid library, e.g., a genomic library or a cDNA library. Examples of genomic libraries include, but are not limited to, whole genome shotgun genomic libraries, reduced representation shotgun genomic libraries, hypomethylated shotgun genomic libraries, hypermethylated shotgun genomic libraries, and 5′ methionine-enriched libraries.

In some embodiments, the sequencing of the first nucleotide sequence and the second nucleotide sequence is carried out without amplifying one or both of the sequences after selecting the host cell based upon the measurable change in expression of the reporter gene. In other embodiments, the sequencing of the first nucleotide sequence and the second nucleotide sequence is carried out without amplifying either sequence after selecting the host cell based upon the measurable change in expression of the reporter gene. In preferred embodiments, “amplifying” refers to non-native forms of amplification of a nucleic acid (e.g., polymerase chain reaction, rolling-circle amplification, or chloramphenicol amplification).

In some embodiments, prior to the sequencing step, the first nucleotide sequence and the second nucleotide sequence are purified from the host cell in the same compartment of a multi-compartment device. For example, prior to sequencing, the first nucleotide sequence and the second nucleotide sequence can be purified from the host cell in the same well of a 96 well vessel.

In some embodiments, sequencing reactions for the first nucleotide sequence and the second nucleotide sequence are carried out in the same compartment of a multi-compartment device, e.g., the same well of a 384 well vessel.

In some embodiments, prior to sequencing, the first nucleotide sequence is purified from the host cell in a first well of a first 96 well vessel and the second nucleotide sequence is purified from the host cell in a second well of a second 96 well vessel, wherein the first and second wells of the first and second 96 well vessels occupy the same relative position in each of the 96 well vessels. The relative positions of the respective wells can be monitored and recorded by a computer connected to a machine that runs the automated processes described herein.

In some embodiments, sequencing reactions for the first nucleotide sequence are carried out in a first well of a first 384 well vessel and sequencing reactions for the second nucleotide sequence are carried out in a second well of a second 384 well vessel, wherein the first and second wells of the first and second 384 well vessels occupy the same relative position in each of the 384 well vessels. The relative positions of the respective wells can be monitored and recorded by a computer connected to a machine that runs the automated processes described herein.

In some embodiments, the method also includes a step of preparing a computer readable record containing an entry which includes a first identifier corresponding to the first polypeptide sequence and a second identifier corresponding to a binding property of the first polypeptide sequence.

In some embodiments, prior to the selecting of the cell based upon the measurable change in expression of the reporter gene, the host cell is placed on a robot compatible substrate which permits automated picking of cells exhibiting the measurable change in expression of the reporter gene. The methods described herein can include and additional step of using an automated device to select host cells exhibiting the measurable change in expression of the reporter gene. For example, bacterial host cells can be deposited on an agar-containing substrate (optionally including an antibiotic that allows for the selection of those cells containing a first fusion protein that binds to a second fusion protein), which is followed by a robotic selection of bacterial colonies that are found to grow on the substrate. Selected colonies can be used to purify and sequence nucleic acids (e.g., plasmids) using the automated methods described herein.

In another aspect, the invention features a method for identifying nucleotide sequences encoding interacting polypeptide sequences, wherein the method includes the steps of: (1) providing a cell population containing 100,000 bacterial host cells, wherein each of the 100,000 bacterial host cells contains a reporter gene operably linked to a transcriptional regulatory sequence which includes a binding site for a DNA-binding domain; (2) introducing into each of the 100,000 bacterial host cells a first chimeric gene encoding a first fusion protein, wherein the first fusion protein contains a first polypeptide sequence and a DNA-binding domain, wherein the first chimeric gene contains a first nucleotide sequence which encodes the first polypeptide sequence, and wherein the first nucleotide sequence is different in the first chimeric gene introduced into each of the 100,000 bacterial host cells; (3) introducing into each of the 100,000 bacterial host cells a second chimeric gene encoding a second fusion protein, wherein the second fusion protein contains a second polypeptide sequence and an activation tag, wherein the second chimeric gene contains a second nucleotide sequence which encodes the second polypeptide sequence, and wherein the second nucleotide sequence is different in the second chimeric gene introduced into each of the 100,000 bacterial host cells; (4) culturing the 100,000 bacterial host cells for a time sufficient to allow an interaction of the first fusion protein and the second fusion protein, if present, wherein the interaction results in a measurable change in expression of the reporter gene; (5) selecting from the 100,000 bacterial host cells those cells that exhibit the measurable change in expression of the reporter gene, to thereby result in selected bacterial host cells; and (6) sequencing the first nucleotide sequence and the second nucleotide sequence contained in selected bacterial host cells, to thereby identify nucleotide sequences encoding interacting polypeptide sequences.

In another aspect, the invention features a method for identifying nucleotide sequences encoding interacting polypeptide sequences, wherein the method includes the steps of: (1) providing a cell population containing a plurality of host cells, wherein each of the plurality of host cells contains a reporter gene operably linked to a transcriptional regulatory sequence which includes a binding site for a DNA-binding domain; (2) introducing into each of the plurality of host cells a first chimeric gene encoding a first fusion protein, wherein the first fusion protein contains a first polypeptide sequence and a DNA binding domain, and wherein the first chimeric gene contains a first nucleotide sequence encoding the first polypeptide sequence; (3) introducing into each of the plurality of host cells a second chimeric gene encoding a second fusion protein, wherein the second fusion protein contains a second polypeptide sequence and an activation tag, wherein the second chimeric gene contains a second nucleotide sequence which encodes the second polypeptide sequence, and wherein the second nucleotide sequence is different in the second chimeric gene introduced into each of the plurality of host cells; (4) culturing the plurality of host cells for a time sufficient to allow an interaction of the first fusion protein and the second fusion protein, if present, wherein the interaction results in a measurable change in expression of the reporter gene; (5) selecting from the plurality of host cells those cells that exhibit the measurable change in expression of the reporter gene, to thereby result in selected host cells; (6) purifying the second nucleotide sequence from each of the selected host cells, wherein the purification is carried out by an automated process in compartments of a multi-compartment device; and (7) sequencing the second nucleotide sequence from each of the selected host cells, to thereby identify nucleotide sequences encoding interacting polypeptide sequences. In some embodiments, the second nucleotide sequences from each of the selected host cells are purified in wells of a 96 well vessel. In some embodiments, sequencing reactions for the second nucleotide sequences are carried out in wells of a 384 well vessel.

In another aspect, the invention features a method for identifying nucleotide sequences encoding interacting polypeptide sequences, wherein the method includes the steps of: (1) providing a cell population containing a plurality of host cells, wherein each of the plurality of host cells contains a reporter gene operably linked to a transcriptional regulatory sequence which includes a binding site for a DNA-binding domain; (2) introducing into each of the plurality of host cells a first chimeric gene encoding a first fusion protein, wherein the first fusion protein contains a first polypeptide sequence and a DNA binding domain, wherein the first chimeric gene contains a first nucleotide sequence which encodes the first polypeptide sequence, and wherein the first nucleotide sequence is different in the first chimeric gene introduced into each of the plurality of host cells; (3) introducing into each of the plurality of host cells a second chimeric gene encoding a second fusion protein, wherein the second fusion protein contains a second polypeptide sequence and an activation tag, and wherein the second chimeric gene contains a second nucleotide sequence encoding the second polypeptide sequence; (4) culturing the plurality of host cells for a time sufficient to allow an interaction of the first fusion protein and the second fusion protein, if present, wherein the interaction results in a measurable change in expression of the reporter gene; (5) selecting from the plurality of host cells those cells that exhibit the measurable change in expression of the reporter gene, to thereby result in selected host cells; (6) purifying the first nucleotide sequence from each of the selected host cells, wherein the purification is carried out by an automated process in compartments of a multi-compartment device; and (7) sequencing the first nucleotide sequence from each of the selected host cells, to thereby identify nucleotide sequences encoding interacting polypeptide sequences. In some embodiments, the first nucleotide sequences from each of the selected host cells are purified in wells of a 96 well vessel. In some embodiments, sequencing reactions for the first nucleotide sequences are carried out in wells of a 384 well vessel.

In another aspect, the invention features a method for identifying nucleotide sequences encoding interacting polypeptide sequences, wherein the method includes the steps of: (1) providing a cell population containing a plurality of host cells, wherein each of the plurality of host cells contains a reporter gene operably linked to a transcriptional regulatory sequence which includes a binding site for a DNA-binding domain; (2) introducing into each of the plurality-of host cells a first chimeric gene encoding a first fusion protein, wherein the first fusion protein contains a first polypeptide sequence and a DNA binding domain, wherein the first chimeric gene contains a first nucleotide sequence which encodes the first polypeptide sequence, and wherein the first nucleotide sequence is different in the first chimeric gene introduced into each of the plurality of host cells; (3) introducing into each of the plurality of host cells a second chimeric gene encoding a second fusion protein, wherein the second fusion protein contains a second polypeptide sequence and an activation tag, wherein the second chimeric gene contains a second nucleotide sequence which encodes the second polypeptide sequence, and wherein the second nucleotide sequence is different in the second chimeric gene introduced into each of the plurality of host cells; (4) culturing the plurality of host cells for a time sufficient to allow an interaction of the first fusion protein and the second fusion protein, wherein the interaction results in a measurable change in expression of the reporter gene; (5) selecting the plurality of host cells based upon the measurable change in expression of the reporter gene; and (6) sequencing the first nucleotide sequence and the second nucleotide sequence contained in each of the plurality of host cells, to thereby identify nucleotide sequences encoding interacting polypeptide sequences.

The methods of the invention can optionally be carried out as detailed below.

The host cell (or plurality of host cells) can be a prokaryotic cell (e.g., a bacterial cell such as Escherichia coli) or a eukaryotic cell (e.g., a yeast cell or a mammalian cell).

The methods described herein can be used for high throughput screening of nucleotide sequences in two-hybrid assays. Accordingly, large numbers of host cells, first chimeric genes, and/or second chimeric genes can be used in such methods.

The plurality of host cells screened in the methods described herein can include at least 100 cells, at least 1,000 cells, at least 10,000 cells, at least 100,000 cells, at least 1,0000,000 cells, at least 10,000,000 cells, at least 100,000,000 cells, at least 1,000,000,000 cells, or more.

The number of different first chimeric genes introduced into the plurality of host cells in the methods described herein can be at least 100, at least 1,000, at least 10,000, at least 100,000, at least 1,0000,000, at least 10,000,000, at least 100,000,000, at least 1,000,000,000, or more.

The number of different second chimeric genes introduced into the plurality of host cells in the methods described herein can be at least 100, at least 1,000, at least 10,000, at least 100,000, at least 1,0000,000, at least 10,000,000, at least 100,000,000, at least 1,000,000,000, or more.

In some embodiments, the numbers of different first and second chimeric genes used in a single two-hybrid assay can vary. For example, the ratio of different first chimeric genes to different second chimeric genes (or vice versa) used in a single assay can be 10 fold, 100 fold, 1,000 fold, 10,000 fold, 100,000 fold, or more. In some embodiments, only one first or second chimeric gene is used to screen against a plurality of first or second chimeric genes.

In some embodiments, a first DNA-binding domain is encoded by the first chimeric gene introduced into a first subset of the plurality of host cells, and a second DNA-binding domain is encoded by the first chimeric gene introduced into a second subset of the plurality of host cells. A third DNA-binding domain can be encoded by the first chimeric gene introduced into a third subset of the plurality of host cells. Additional DNA-binding domains, e.g., four, five, six, or more can be used in the methods described herein.

In some embodiments, the DNA-binding domain is fused to the amino terminus of the first polypeptide sequence in a first subset of the plurality of host cells, and the DNA-binding domain is fused to the carboxy terminus of the first polypeptide sequence in a second subset of the plurality of host cells.

In some embodiments, a first activation tag is encoded by the second chimeric gene introduced into a first subset of the plurality of host cells, and a second activation tag is encoded by the second chimeric gene introduced into a second subset of the plurality of host cells. A third activation tag can be encoded by the second chimeric gene introduced into a third subset of the plurality of host cells. Additional activation tags, e.g., four, five, six, or more can be used in the methods described herein.

In some embodiments, the activation tag is fused to the amino terminus of the second polypeptide sequence in a first subset of the plurality of host cells, and the activation tag is fused to the carboxy terminus of the second polypeptide sequence in a second subset of the plurality of host cells.

In those embodiments that include two or more different DNA-binding domains and/or activation tags and/or two or more different configurations of the same DNA-binding domain and/or activation tag (e.g., amino or carboxy terminus), the detection of the same binding interaction in the context of two or more different fusion proteins can be used as a confirmation of the relevance of the binding event (e.g., to reduce the likelihood that a given interaction is an artifact of the assay system used).

The first and/or second nucleotide sequence can be derived from a nucleic acid library, e.g., a genomic library or a cDNA library. Examples of genomic libraries include, but are not limited to, whole genome shotgun genomic libraries, reduced representation shotgun genomic libraries, hypomethylated shotgun genomic libraries, hypermethylated shotgun genomic libraries, and 5′ methionine-enriched libraries.

In some embodiments, prior to the sequencing step, the first nucleotide sequence and the second nucleotide sequence for each of the plurality of host cells are purified in the same compartment of a multi-compartment device. For example, prior to sequencing, the first nucleotide sequence and the second nucleotide sequence can be purified from a host cell in the same well of a 96 well vessel.

In some embodiments, sequencing reactions for the first nucleotide sequence and the second nucleotide sequence for each of the plurality of host cells are carried out in the same compartment of a multi-compartment device, e.g., the same well of a 384 well vessel.

In some embodiments, prior to sequencing, the first nucleotide sequence for each of the plurality of host cells is purified from the host cell in a first well of a first 96 well vessel and the second nucleotide for each of the plurality of host cells sequence is purified from the host cell in a second well of a second 96 well vessel, wherein the first and second wells of the first and second 96 well vessels occupy the same relative position in each of the 96 well vessels. The relative positions of the respective wells can be monitored and recorded by a computer connected to a machine that runs the automated processes described herein.

In some embodiments, sequencing reactions for the first nucleotide sequence for each of the plurality of host cells are carried out in a first well of a first 384 well vessel and sequencing reactions for the second nucleotide sequence for each of the plurality of host cells are carried out in a second well of a second 384 well vessel, wherein the first and second wells of the first and second 384 well vessels occupy the same relative position in each of the 384 well vessels. The relative positions of the respective wells can be monitored and recorded by a computer connected to a machine that runs the automated processes described herein.

In some embodiments, prior to the selecting of the cell based upon the measurable change in expression of the reporter gene, the plurality of host cells are placed on a robot compatible substrate which permits automated picking of cells exhibiting the measurable change in expression of the reporter gene. The methods described herein can include and additional step of using an automated device to select host cells exhibiting the measurable change in expression of the reporter gene. For example, bacterial host cells can be deposited on an agar-containing substrate (optionally including an antibiotic that allows for the selection of those cells containing a first fusion protein that binds to a second fusion protein), which is followed by a robotic selection of bacterial colonies that are found to grow on the substrate. Selected colonies can be used to purify and sequence nucleic acids (e.g., plasmids) using the automated methods described herein.

In some embodiments, after selecting host cells based upon the measurable change in expression of the reporter gene, the first nucleotide sequence and the second nucleotide sequence are purified from the plurality of host cells and sequenced without amplification of the sequences prior to the carrying out of the sequencing step.

In some embodiments, the sequencing includes single-plex sequencing of the first nucleotide sequence and the second nucleotide sequence.

In some embodiments, sequencing reactions are carried out in the wells of 384 well vessels using a reaction volume of 25, 15, 10, 7, 5 μl or less.

In some embodiments, reaction products of sequencing reactions are transferred directly from the well of a multi-compartment device, e.g., a 384 well vessel, to a capillary, microfabricated, or single molecule DNA sequencer.

In some embodiments of the methods described herein, the invention also includes shotgun sequencing a nucleic acid library or the genome of an organism or a virus, wherein the method identifies in serial or in parallel via a two-hybrid assay the protein-protein interactions of polypeptides encoded by the nucleic acid library or the genome of the organism or virus. In such methods, nucleic acid vectors used to carry out the shotgun sequencing can be the same as the nucleic acid vectors used to carry out the two-hybrid assay.

For example, a genomic library can be inserted in a given nucleic acid vector, which nucleic acid vector is then used in automated high throughput methods of sequencing the genome. These methods can identify sequences as containing likely open reading frames, which sequences can subsequently be used in a two hybrid assay described herein. Accordingly, one or more sequences identified by the shotgun sequencing methods can be used in a two hybrid assay to screen against a plurality of target sequences (e.g., the two hybrid assay can be carried out against the genomic library, using the same vector used for the shotgun sequencing methods).

In some embodiments, prior to sequencing of the a nucleotide sequence or nucleotide sequences as described herein, a selected host cell or selected host cells are grown in compartments of a multi-compartment device. For example, such a device can have 24, 48, 96, 384, 1536 or more compartments. Preferably, the number of compartments of the device is divisible by 24, 48, and/or 96. Following the culture of a selected host cell in such a compartment, nucleotide sequences contained in a selected host cell can be purified, optionally in the same compartment, prior to sequencing.

In some embodiments, the methods include a step of preparing a computer readable record including a plurality of entries, each entry containing a first identifier which corresponds to a first polypeptide sequence and a second identifier which corresponds to a binding property of the first polypeptide sequence. For example, the second identifier can indicate whether the first polypeptide sequence binds to a second polypeptide sequence. Identifiers can also indicate additional polypeptides to which the first polypeptide binds. In addition, the computer readable record can include additional entries that describe, e.g., an activity or structure (e.g., domain structure) of the first polypeptide sequence and/or an activity or structure of a polypeptide to which the first polypeptide sequence binds.

In some embodiments, at least one combination of first and second chimeric genes is used as a positive control in the methods described herein. In such methods, the first and second chimeric genes encode first and second polypeptide sequences that are known to interact with one another. Accordingly, introducing such chimeric genes into the plurality of host cells can be used as a positive control to confirm that the high throughput screening and sequencing methods described herein are being carried out under conditions that allow for the detection of “positive” events.

In some embodiments, populations of nucleic acids encoding first and/or second fusion proteins are supplied by a first party, e.g., a customer, to a second party, e.g., a party conducting a two-hybrid assay. Such methods can further include steps of providing information about the binding of the first and/or second fusion proteins to the first party from the second party. Such information can be provided in a computer readable form, e.g., as described herein with respect to pluralities of entries containing identifiers characterizing polypeptide interactions detected in two-hybrid screens.

In other aspects, the invention includes methods of providing sets of protein interactions by: (1) carrying out a method as described herein to characterize the binding of one or more first polypeptide sequences to one or more second polypeptide sequences, to result in a record of protein interactions detected in a first screen; (2) carrying out a method as described herein to characterize the binding of one or more first polypeptide sequences to one or more second polypeptide sequences, to result in a record of protein interactions detected in a second screen; and (3) creating a record, e.g., a computer readable record, that identifies protein interactions detected in the first screen and/or the second screen. The computer readable record can contain entries as described herein for the polypeptides identified in the screens. The first and second screens can be carried out using similar or different nucleic acid sources, e.g., genomic or cDNA libraries as described herein. Additional screens can be carried be carried out, wherein the protein interactions detected in such screens are used to construct the record in step (3) of the method.

In other aspects, the invention includes methods including steps of: (1) providing a record of sets of protein interactions as described herein; (2) accepting a query from a first party as to whether a polypeptide has a selected property, e.g., a binding property; (3) searching the record of sets of protein interactions to determine whether the polypeptide has the selected property; and (4) providing an answer to the first party as to whether the polypeptide has the selected property.

As used herein, a “DNA-binding domain” is an amino acid sequence that is capable of directing the binding of a polypeptide to a specific DNA sequence. A DNA-binding domain can be derived from naturally occurring proteins or from artificial sequences.

As used herein, an “activation tag” is an amino acid sequence that participates as a component of an RNA polymerase or recruits an active polymerase complex. For example, an activation tag can be a polymerase interaction domain or some other polypeptide sequence that interacts with, or is covalently bound to, one or more subunits (or a fragment thereof) of an RNA polymerase complex. An activation tag can also be an amino acid sequence that is derived from a transcription factor or another protein that interacts, directly or indirectly, with a polymerase complex. An activation tag can be derived from a naturally occurring protein or from an artificial sequence, e.g., from a random polypeptide library.

As used herein, a “reporter gene” is any gene whose expression can be assayed, so as to detect a measurable change in expression of the gene. A reporter gene can be any gene that expresses a detectable gene product, e.g., RNA or protein. For example, a reporter gene can encode a protein that provides a phenotypic marker, e.g., a protein that is necessary for cell growth or a toxic protein leading to cell death (e.g., a protein that confers antibiotic resistance or complements an auxotrophic phenotype), a protein detectable by a colorimetric/fluorometric assay leading to the presence or absence of color/fluorescence, or a protein providing a surface antigen for which specific antibodies/ligands are available.

A reporter gene can be included in a construct in the form of a fusion gene with a gene that includes the reporter gene operably linked to transcriptional regulatory sequences which include a binding site for a DNA-binding domain. The ability of the transcriptional regulatory sequences to direct transcription of the reporter gene is dependent upon a transcriptional complex being recruited by virtue of an interaction between bait and prey fusion proteins. The transcriptional regulatory sequences can include a promoter and other regulatory regions that modulate the activity of the promoter, or regulatory sequences that modulate the activity or efficiency of the RNA polymerase that recognizes the promoter. The reporter gene construct includes a nucleotide sequence that is specifically bound by the DNA-binding domain of the bait fusion protein. This binding site for the DNA-binding domain is located sufficiently proximal to the promoter sequence of the reporter gene so as to cause increased reporter gene expression upon recruitment of an RNA polymerase complex by a bait fusion protein bound at the binding site for the DNA-binding domain.

As used herein, “operably linked” refers to gene and transcriptional regulatory sequences being connected in such a way as to permit expression of the gene in a manner dependent upon factors interacting with the transcriptional regulatory sequences. For example, the binding site for the DNA-binding domain is operably linked to the reporter gene such that transcription of the reporter gene will be dependent, at least in part, upon bait-prey complexes bound to the binding site for the DNA-binding domain.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

The present invention provides methods for determining the sequence of nucleic acids encoding interacting polypeptide sequences. The methods described herein can be used to determine the sequence of nucleic acids contained in a population of nucleic acids, while specifically identifying those sequences contained in the population that encode interacting polypeptide sequences. According to these methods, a two hybrid assay is carried out to select candidates for sequencing that may encode interacting proteins. Accordingly, these methods allow for the creation of an “interaction map” of, for example, the genome of an organism or for the genes expressed by a particular tissue. The nature of the “interaction map” depends upon the nucleic acid sequences that are used in the screening assays described herein.

Two-Hybrid Assays

The invention encompasses methods of determining the nucleotide sequences of nucleic acids encoding interacting protein sequences. The methods of the invention use two-hybrid assays to select nucleic acids that are to be sequenced as described herein. In general, a two hybrid assay is carried out to select cells wherein a first fusion protein (bait) binds to a second fusion protein (prey). Once cells have been selected that have undergone such a binding event, the nucleotide sequences encoding the portions of the fusion proteins responsible for the interaction are isolated and sequenced. Methods of performing two-hybrid assays are well known to those of skill in the art and are described in detail in, for example, U.S. Pat. No. 5,667,973 and U.S. Pat. No. 5,925,523. The entire content of these references is incorporated by reference.

Reporter Genes

The reporter gene used in the methods of the invention permits the detection of transcriptional modulation that results from the interaction of a bait and a prey protein in a host cell. Accordingly, a reporter gene construct can be inserted into a host cell to generate a detection signal dependent on the interaction of the bait and prey fusion proteins. Typically, the reporter gene construct includes a reporter gene operably linked with one or more transcriptional regulatory elements which include, or are linked to, a binding site for the DNA-binding domain of the bait fusion protein, with the level of expression of the reporter gene providing the prey protein interaction-dependent detection signal. Many reporter genes and transcriptional regulatory elements useful in two hybrid assays are known to those of skill in the art. Moreover, binding sites are known for a wide variety of DNA-binding domains that can used to construct the bait proteins of the present invention. Examples of binding sites for DNA-binding domains include the lambda operator, the LexA operator, and the pho box.

Examples of reporter genes include, but are not limited to chloramphenicol acetyl transferase, luciferase, beta-galactosidase, firefly luciferase, bacterial luciferase, phycobiliproteins, green fluorescent protein, alkaline phosphatase, and secreted alkaline phosphatase. Other examples of suitable reporter genes include those that encode proteins conferring drug/antibiotic resistance to a host cell (e.g., a host bacterial cell) or that encode proteins required to complement an auxotrophic phenotype.

Transcription from the reporter gene may be measured using any method known to those of skill in the art to be suitable. For example, specific mRNA expression may be detected using Northern blots or specific protein product may be identified by a characteristic stain or an intrinsic activity.

In some embodiments, the product of the reporter gene is detected by an intrinsic activity associated with that product. For instance, the reporter gene may encode a gene product that, by enzymatic activity, gives rise to a detection signal based on color, fluorescence, or luminescence.

In other embodiments, the reporter gene allows for a selection such that cells in which the reporter gene is activated have a growth advantage. For example the reporter could enhance cell viability, e.g., by relieving a cell nutritional requirement and/or provide resistance to a drug (e.g., an antibiotic). For example the reporter gene can encode a gene product that confers the ability to grow in the presence of a selective agent, e.g., chloramphenicol or kanamycin. In bacteria, suitable positively selectable genes include genes involved in biosynthesis or drug resistance.

In other embodiments, the reporter gene can encode a cell surface protein for which antibodies or ligands are available. Expression of the reporter gene allows cells to be detected or affinity purified by the presence of the surface protein.

Host Cells

The methods of the invention can be performed using prokaryotic cells (e.g., bacterial cells) or eukaryotic cells (e.g., yeast or mammalian cells).

Exemplary prokaryotic host cells include bacterial strains of Escherichia (e.g., Escherichia coli), Bacillus (e.g., Bacillus subtilis), Streptomyces, Pseudomonas, Salmonella, Serratia, and Shigella. Techniques for transforming these hosts and expressing foreign genes cloned in them are well known in the art. Vectors used for expressing foreign genes in bacterial hosts will generally contain a selectable marker, such as a gene for antibiotic resistance, and a promoter which functions in the host cell. Appropriate promoters include trp and phage gamma promoter systems. Plasmids useful for transforming bacteria include pBR322, the pUC plasmids, pCQV2, pACYC plasmids, pRW plasmids, and derivatives thereof.

Bait Constructs (First Chimeric Gene)

The methods of the invention include the introduction into a host cell of a “first chimeric gene” which encodes a first fusion protein (also termed a “bait” protein). The first fusion protein contains a first polypeptide sequence and a DNA-binding domain. The first chimeric gene is constructed by ligating a nucleic acid encoding a DNA-binding domain to a first nucleotide sequence that encodes the first polypeptide sequence.

The use of recombinant DNA techniques to create the first chimeric gene is well known in the art. For example, the joining of various nucleic acids can be performed in accordance with conventional techniques, employing, e.g., blunt-ended or stagger-ended termini for ligation, restriction enzyme digestion to provide for appropriate termini, filling in of cohesive ends as appropriate alkaline phosphatase treatment to avoid undesirable joining, and enzymatic ligation. In addition, site directed recombination (e.g., Cre/lox site-specific recombination technology) can be used to create chimeric gene constructs described herein.

Any source of nucleic acids can be used to obtain the first nucleotide sequence. For example, the first nucleotide sequence can be derived from a nucleic acid library, e.g., a genomic or cDNA library as described herein. In such methods, the identity of the first nucleotide sequence need not be known before the carrying out of a two hybrid assay. Rather, the interaction of the first fusion protein (bait) with the second fusion protein (prey) is used to identify host cells that contain two nucleotide sequences that encode protein sequences that interact with each other. Following the selection of the first and second nucleotide sequences in a two hybrid assay, the nucleotide sequences can then be isolated and sequenced. Such methods permit the selection and identification of nucleotide sequences contained in a population of nucleic acids (e.g., a whole genome library) that encode interacting polypeptides.

DNA-binding domains are well known and include, but are not limited to such motifs as helix-turn-helix motifs (such as found in lambda cI), winged helix-turn helix motifs (such as found in certain heat shock transcription factors), and/or zinc fingers/zinc clusters.

The DNA-binding domain portion of the first fusion protein can be derived using all, or a DNA-binding portion, of a transcriptional regulatory protein, e.g., of a transcriptional activator or transcriptional repressor, that retains the ability to selectively bind to specific nucleotide sequences. The DNA-binding domains of the bacteriophage lambda cI protein and the E. coli LexA repressor represent exemplary DNA binding domains that can be included in the first fusion protein used in two hybrid assays as described herein.

The DNA-binding domain used to generate the first fusion protein can include oligomerization motifs. Certain transcriptional regulators dimerize, with dimerization promoting cooperative binding of the two monomers to their cognate recognition elements. For example, where the first protein includes a LexA-DNA binding domain, it can further include a LexA dimerization domain. Other oligomerization motifs useful in the methods of the invention will be readily recognized by those skilled in the art. Exemplary oligomerization motifs include the oligomerization domain of lambda cI.

In some embodiments, a polypeptide linker is inserted between the DNA-binding domain of the first fusion protein and the first polypeptide sequence. Where the first fusion protein also includes oligomerization sequences, it may be preferable to situate the linker between the oligomerization sequences and the first polypeptide sequence. The linker can facilitate enhanced flexibility of the fusion protein allowing the DNA-binding domain to freely interact with a DNA binding site, and, if present, the oligomerization sequences to make inter-protein contacts. The linker can also reduce steric hindrance between the two fragments, and allow appropriate interaction of the first polypeptide sequence with a second polypeptide sequence contained in the second fusion protein. The linker can be of natural origin, such as a sequence determined to exist in random coil between two domains of a protein. Alternatively, the linker can be of synthetic origin. Exemplary linkers are described in Huston et al. (1988) PNAS 85:4879; and U.S. Pat. No. 5,091,513.

Prey Constructs (Second Chimeric Gene)

The methods of the invention include the introduction into a host cell of a “second chimeric gene” which encodes a second fusion protein (also termed a “prey” protein). The second fusion protein contains a second polypeptide sequence and an activation tag. As detailed above with respect to the first chimeric gene, the second chimeric gene is constructed by ligating a nucleic acid encoding an activation tag to a second nucleotide sequence that encodes the second polypeptide sequence.

As described herein, the second polypeptide sequence of the second fusion protein (prey) is capable of binding to the first polypeptide sequence of the first fusion protein (bait). Any source of nucleic acids can be used to obtain the second nucleotide sequence, which encodes the second polypeptide sequence. For example, the second nucleotide sequence can be derived from a nucleic acid library, e.g., a genomic or cDNA library as described herein. In the methods of the invention, the identity of the second nucleotide sequence (and the first nucleotide sequence) need not be known before the carrying out of a two hybrid assay. Rather, the interaction of the first fusion protein (bait) with the second fusion protein (prey) is used to identify host cells that contain two nucleotide sequences that encode protein sequences that interact with each other. Following the selection of the first and second nucleotide sequences in a two hybrid assay, the nucleotide sequences can then be isolated and sequenced. Such methods permit the selection and identification of nucleotide sequences contained in a population of nucleic acids (e.g., a whole genome library) that encode interacting polypeptides.

The activation tag of the second fusion protein can be, for example, all or a portion of an RNA polymerase subunit, such as the polymerase interaction domain of the N-terminal domain of the RNA polymerase a subunit. In such examples, protein-protein contact between the first fusion protein and second fusion protein (via an interaction of the first and second polypeptide sequences of the proteins) links the DNA-binding domain of the first fusion protein with the polymerase interaction domain of the second fusion protein, generating a protein complex capable of directly recruiting a functional RNA polymerase enzyme to DNA sequences proximate to the DNA bound first fusion protein.

In one embodiment, the second fusion protein includes a sufficient portion of the amino-terminal domain of the a subunit of an RNA polymerase to permit assembly of transcriptionally active RNA polymerase complexes that include the second fusion protein. The alpha subunit, which initiates the assembly of RNA polymerase by forming a dimer, has two independently folded domains. The larger amino-terminal domain mediates dimerization and the subsequent assembly of the polymerase complex. The second polypeptide sequence can be fused in frame to the alpha-NTD or a fragment thereof that retains the ability to assemble a functional RNA polymerase complex.

The methods of the present invention include the use of polymerase interaction domains containing portions of other RINA polymerase subunits or portions of molecules which associate with an RNA polymerase subunit or subunits.

The second fusion proteins used in the methods described herein can differ in the polymerase interaction domains or target surfaces they include, and in whether they contain other useful moieties such as epitope tags or oligomerization domains.

In some embodiments, a polypeptide linker is inserted between the activation tag of the second fusion protein and the second polypeptide sequence. The linker can facilitate enhanced flexibility of the fusion protein allowing the activation tag to freely interact with target proteins and/or nucleic acid sequences. The linker can also reduce steric hindrance between the two fragments, and allow appropriate interaction of the second polypeptide sequence with a first polypeptide sequence contained in the first fusion protein. The linker can be of natural origin, such as a sequence determined to exist in random coil between two domains of a protein. Alternatively, the linker can be of synthetic origin. Exemplary linkers are described in Huston et al. (1988) PNAS 85:4879; and U.S. Pat. No. 5,091,513.

Sources of Nucleotide Sequences for Two-Hybrid Assays

The methods of the invention can use any source of nucleic acid for introduction of the first and second nucleotide sequences into the fusion gene constructs described herein. In general, the identity of the nucleotide sequence is unknown prior to the carrying out of the two hybrid assay. Accordingly, a given nucleotide sequence is sequenced following a “positive result” in the two hybrid assay, so as to determine the identity of the sequence that induced such a positive result.

A nucleic acid library can be used as the source of the first and/or second nucleotide sequence. Examples of nucleic acid libraries include genomic libraries and cDNA libraries. In some cases, the same library is used to generate both the plurality of first chimeric genes as well as the plurality of second chimeric genes. In such cases, the results of the two hybrid assay and the sequencing information derived therefrom can be used to create an interaction map for a given tissue (if a cDNA library is used) or for a given genome (if a genomic library is used). Such an interaction map profiles and catalogues the nucleic acids expressed by a given tissue or contained in a given genome that encode functional proteins, as determined by their ability to interact with other proteins encoded by nucleic acids in the same library.

Examples of genomic libraries include, but are not limited to, whole genome shotgun genomic libraries, reduced representation shotgun genomic libraries, hypomethylated shotgun genomic libraries, hypermethylated shotgun genomic libraries, and 5′ methionine-enriched libraries. A genomic library can be constructed to enrich for sequences that are likely associated with genes or that contain open reading frames or to remove sequences that are likely to contain noncoding DNA.

In those embodiments where cDNA libraries are used, cDNAs can be constructed from any mRNA population. Such a library of choice may be constructed using commercially available kits or using well established preparative procedures (see, e.g., Ausubel et al., Current Protocols in Molecular Biology, John Wiley & Sons, Inc., 2002). In addition, a number of cDNA libraries (from a number of different organisms) are publicly and commercially available.

Isolation and Sequencing of Nucleotide Sequences

Following the selection of host cells that contain interacting bait and prey fusion proteins, the nucleotide sequences encoding the first and second polypeptide sequences are isolated and sequenced. Methods for isolating nucleic acids from eukaryotic and prokaryotic cells and sequencing the isolated nucleic acids are well known to those of skill in the art (see, e.g., Ausubel et al., Current Protocols in Molecular Biology, John Wiley & Sons, Inc., 2002). Additional methods for isolating nucleic acids are described in U.S. application Ser. No. 10/042,923, filed Jan. 9, 2002.

In some embodiments, high-throughput automated sequencing methods are used to determine the sequences of the first and second nucleotide sequences. Accordingly, purification of the nucleotide sequences can be carried out in multi-well vessels, such as 96 well plates, using automated processes. In addition, sequencing reactions can be carried out in multi-well vessels, such as 384 well plates, also using automated processes. The first and second nucleotide sequences can be purified in the same well or in different wells of a multi-well vessel. If the first and second nucleotide sequences are purified in different wells, then an association between the different wells must be maintained so that once the sequence information is obtained, it can be recorded that the first nucleotide sequence encodes a first polypeptide sequence that interacts with a second polypeptide sequence encoded by the second nucleotide sequence. In addition, associations must be maintained between the well used to purify a nucleotide sequence (e.g., a well of a 96 well plate) and the wells used to carry out sequencing reactions on the nucleotide sequence (e.g., a well of a 384 well plate). Such associations between different wells can be maintained by a computer connected to a machine that runs the automated processes described herein.

In general, the sequencing reactions for the first and second nucleotide sequences are carried out using the same vector, e.g., a plasmid, that was used to perform the two hybrid screen. In such embodiments, the nucleotide sequences are not excised from the bait or prey vectors after the two hybrid screen, prior to sequencing. Rather, primers for the sequencing reactions are designed to anneal to target sequences contained in each of the constructs encoding the first and second chimeric genes.

As the methods of the invention are directed to high-throughput screens for identifying nucleotide sequences encoding interacting polypeptides, the invention includes methods that do not entail the amplification of a given nucleotide sequence following the selection of a host cell. Avoiding such an amplification step allows for host cells, e.g., bacterial cells, to be selected and be subjected to sequence analysis immediately following the isolation of the relevant nucleotide sequences. Such methods avoid the additional steps and expenses associated with isolation of colonies and amplification of target sequences prior to sequencing.

High-throughput automated whole genome sequencing methods that can be used to sequence nucleic acids selected by the two-hybrid assays described herein are detailed in, e.g., Lander et al. (2001) Nature 409:860-921, Venter et al. (2001) Science 291:1304-1351, Siegel et al. (1999) Genome Res. 9:297-307, Weber et al. (1997) Genome Res. 7:401-409, Green (1997) Genome Res. 7:410-417, and Adams et al. (1944) Automated DNA Sequencing and Analysis, Academic Press.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying nucleotide sequences encoding interacting polypeptide sequences, the method comprising:
providing a host cell containing a reporter gene operably linked to a transcriptional regulatory sequence which includes a binding site for a DNA-binding domain;
introducing into the host cell a first chimeric gene encoding a first fusion protein, wherein the first fusion protein comprises a first polypeptide sequence and a DNA binding domain, and wherein the first chimeric gene comprises a first nucleotide sequence encoding the first polypeptide sequence;
introducing into the host cell a second chimeric gene encoding a second fusion protein, wherein the second fusion protein comprises a second polypeptide sequence and an activation tag, and wherein the second chimeric gene comprises a second nucleotide sequence encoding the second polypeptide sequence;
culturing the host cell for a time sufficient to allow an interaction of the first fusion protein and the second fusion protein, wherein the interaction results in a measurable change in expression of the reporter gene;
selecting the host cell based upon the measurable change in expression of the reporter gene; and
sequencing the first nucleotide sequence and the second nucleotide sequence, to thereby identify nucleotide sequences encoding interacting polypeptide sequences.

2. The method of claim 1, wherein the host cell is a prokaryotic cell.

3. The method of claim 1, wherein the host cell is a eukaryotic cell.

4. The method of claim 1, wherein the first nucleotide sequence is derived from a nucleic acid library.

5. The method of claim 1, wherein the second nucleotide sequence is derived from a nucleic acid library.

6. The method of claim 1, wherein the first nucleotide sequence and the second nucleotide sequence are derived from nucleic acid libraries.

7. The method of claim 6, wherein the nucleic acid libraries are genomic DNA libraries.

8. The method of claim 7, wherein the genomic DNA libraries are whole genome shotgun genomic libraries.

9. The method of claim 7, wherein the genomic DNA libraries are reduced representation shotgun genomic libraries.

10. The method of claim 7, wherein the genomic DNA libraries are hypomethylated shotgun genomic libraries.

11. The method of claim 7, wherein the genomic DNA libraries are hypermethylated shotgun genomic libraries.

12. The method of claim 6, wherein the nucleic acid libraries are cDNA libraries.

13. The method of claim 6, wherein the nucleic acid libraries are 5′ methionine-enriched DNA libraries.

14. The method of claim 1, wherein the sequencing of the first nucleotide sequence and the second nucleotide sequence is carried out without amplifying one or both of the sequences after selecting the host cell based upon the measurable change in expression of the reporter gene.

15. The method of claim 1, wherein the sequencing of the first nucleotide sequence and the second nucleotide sequence is carried out without amplifying either sequence after selecting the host cell based upon the measurable change in expression of the reporter gene.

16. The method of claim 15, wherein, prior to sequencing, the first nucleotide sequence and the second nucleotide sequence are purified from the host cell in the same compartment of a multi-compartment device.

17. The method of claim 15, wherein, prior to sequencing, the first nucleotide sequence and the second nucleotide sequence are purified from the host cell in the same well of a 96 well vessel.

18. The method of claim 16, wherein sequencing reactions for the first nucleotide sequence and the second nucleotide sequence are carried out in the same well of a 384 well vessel.

19. The method of claim 15, wherein, prior to sequencing, the first nucleotide sequence is purified from the host cell in a first well of a first 96 well vessel and the second nucleotide sequence is purified from the host cell in a second well of a second 96 well vessel, wherein the first and second wells of the first and second 96 well vessels occupy the same relative position in each of the 96 well vessels.

20. The method of claim 19, wherein sequencing reactions for the first nucleotide sequence are carried out in a first well of a first 384 well vessel and sequencing reactions for the second nucleotide sequence are carried out in a second well of a second 384 well vessel, wherein the first and second wells of the first and second 384 well vessels occupy the same relative position in each of the 384 well vessels.

21. The method of claim 1, further comprising preparing a computer readable record comprising an entry which includes a first identifier corresponding to the first polypeptide sequence and a second identifier corresponding to a binding property of the first polypeptide sequence.

22. The method of claim 1, wherein prior to the selecting of the cell based upon the measurable change in expression of the reporter gene, the host cell is placed on a robot compatible substrate which permits automated picking of cells exhibiting the measurable change in expression of the reporter gene.

23. The method of claim 22, further comprising using an automated device to select the host cell exhibiting the measurable change in expression of the reporter gene.

24. A method for identifying nucleotide sequences encoding interacting polypeptide sequences, the method comprising:
providing a cell population comprising a plurality of host cells, wherein each of the plurality of host cells contains a reporter gene operably linked to a transcriptional regulatory sequence which includes a binding site for a DNA-binding domain;
introducing into each of the plurality of host cells a first chimeric gene encoding a first fusion protein, wherein the first fusion protein comprises a first polypeptide sequence and a DNA binding domain, wherein the first chimeric gene comprises a first nucleotide sequence which encodes the first polypeptide sequence, and wherein the first nucleotide sequence is different in the first chimeric gene introduced into each of the plurality of host cells;
introducing into each of the plurality of host cells a second chimeric gene encoding a second fusion protein, wherein the second fusion protein comprises a second polypeptide sequence and an activation tag, wherein the second chimeric gene comprises a second nucleotide sequence which encodes the second polypeptide sequence, and wherein the second nucleotide sequence is different in the second chimeric gene introduced into each of the plurality of host cells;
culturing the plurality of host cells for a time sufficient to allow an interaction of the first fusion protein and the second fusion protein, wherein the interaction results in a measurable change in expression of the reporter gene;
selecting the plurality of host cells based upon the measurable change in expression of the reporter gene; and
sequencing the first nucleotide sequence and the second nucleotide sequence contained in each of the plurality of host cells, to thereby identify nucleotide sequences encoding interacting polypeptide sequences.

25. The method of claim 24, wherein the plurality of host cells comprises at least 100 cells.

26. The method of claim 25, wherein the plurality of host cells comprises at least 1,000 cells.

27. The method of claim 26, wherein the plurality of host cells comprises at least 10,000 cells.

28. The method of claim 27, wherein the plurality of host cells comprises at least 100,000 cells.

29. The method of claim 28, wherein the plurality of host cells comprises at least 1,0000,000 cells.

30. The method of claim 29, wherein the plurality of host cells comprises at least 10,000,000 cells.

31. The method of claim 30, wherein the plurality of host cells comprises at least 100,000,000 cells.

32. The method of claim 31, wherein the plurality of host cells comprises at least 1,000,000,000 cells.

33. The method of claim 24, wherein at least 1,000 different first chimeric genes are introduced into the plurality of host cells.

34. The method of claim 33, wherein at least 10,000 different first chimeric genes are introduced into the plurality of host cells.

35. The method of claim 34, wherein at least 100,000 different first chimeric genes are introduced into the plurality of host cells.

36. The method of claim 24, wherein at least 1,000 different second chimeric genes are introduced into the plurality of host cells.

37. The method of claim 36, wherein at least 10,000 different second chimeric genes are introduced into the plurality of host cells.

38. The method of claim 37, wherein at least 100,000 different second chimeric genes are introduced into the plurality of host cells.

39. The method of claim 26, wherein the plurality of host cells are prokaryotic cells.

40. The method of claim 26, wherein the plurality of host cells are eukaryotic cells.

41. The method of claim 26, wherein a first DNA-binding domain is encoded by the first chimeric gene introduced into a first subset of the plurality of host cells, and wherein a second DNA-binding domain is encoded by the first chimeric gene introduced into a second subset of the plurality of host cells.

42. The method of claim 41, wherein a third DNA-binding domain is encoded by the first chimeric gene introduced into a third subset of the plurality of host cells.

43. The method of claim 26, wherein the DNA-binding domain is fused to the amino terminus of the first polypeptide sequence in a first subset of the plurality of host cells, and wherein the DNA-binding domain is fused to the carboxy terminus of the first polypeptide sequence in a second subset of the plurality of host cells.

44. The method of claim 26, wherein a first activation tag is encoded by the second chimeric gene introduced into a first subset of the plurality of host cells, and wherein a second activation tag is encoded by the second chimeric gene introduced into a second subset of the plurality of host cells.

45. The method of claim 44, wherein a third activation tag is encoded by the second chimeric gene introduced into a third subset of the plurality of host cells.

46. The method of claim 39, wherein the first nucleotide sequence is derived from a nucleic acid library.

47. The method of claim 39, wherein the second nucleotide sequence is derived from a nucleic acid library.

48. The method of claim 39, wherein the first nucleotide sequence and the second nucleotide sequence are derived from nucleic acid libraries.

49. The method of claim 48, wherein the nucleic acid libraries are genomic DNA libraries.

50. The method of claim 49, wherein the genomic DNA libraries are whole genome shotgun genomic libraries.

51. The method of claim 49, wherein the genomic DNA libraries are reduced representation shotgun genomic libraries.

52. The method of claim 49, wherein the genomic DNA libraries are hypomethylated shotgun genomic libraries.

53. The method of claim 49, wherein the genomic DNA libraries are hypermethylated shotgun genomic libraries.

54. The method of claim 48, wherein the nucleic acid libraries are cDNA libraries.

55. The method of claim 48, wherein the nucleic acid libraries are 5′ methionine-enriched DNA libraries.

56. The method of claim 26, wherein the sequencing of the first nucleotide sequence and the second nucleotide sequence in each of the host cells is carried out without amplifying either sequence after selecting the plurality of host cells based upon the measurable change in expression of the reporter gene.

57. The method of claim 56, wherein, prior to sequencing, the first nucleotide sequence and the second nucleotide sequence for each of the plurality of host cells are purified from the respective host cells in the same well of a 96 well vessel.

58. The method of claim 57, wherein sequencing reactions for the first nucleotide sequence and the second nucleotide sequence for each of the plurality of host cells are carried out in the same well of a 384 well vessel.

59. The method of claim 26, wherein prior to selecting of the cells based upon the measurable change in expression of the reporter gene, the plurality of host cells are placed on a robot compatible substrate which permits automated picking of cells exhibiting the measurable change in expression of the reporter gene.

60. The method of claim 59, further comprising using an automated device to pick the plurality of host cells exhibiting the measurable change in expression of the reporter gene.

61. The method of claim 60, wherein the plurality of host cells are prokaryotic cells.

62. The method of claim 61, wherein after selecting of the cells based upon the measurable change in expression of the reporter gene, the first nucleotide sequence and the second nucleotide sequence are purified from the plurality of host cells and sequenced without amplification of the sequences prior to the carrying out of the sequencing step.

63. The method of claim 62, wherein the first nucleotide sequence and the second nucleotide sequence of each of the plurality of host cells are purified in the same vessel.

64. The method of claim 63, wherein the first nucleotide sequence and the second nucleotide sequence of the plurality of host cells are purified in wells of 96 well vessels.

65. The method of claim 64, wherein sequencing reactions for each of the first nucleotide sequence and the second nucleotide sequence of the plurality of host cells are carried out in wells of 384 well vessels.

66. The method of claim 64, wherein the sequencing comprises single-plex sequencing of the first nucleotide sequence and the second nucleotide sequence.

67. The method of claim 64, wherein the sequencing reactions in the wells of the 384 well vessels is carried out using a reaction volume of 25 μl or less.

68. The method of claim 64, wherein reaction products of the sequencing reactions are transferred directly from the well of the 384 well vessel to a capillary, microfabricated, or single molecule DNA sequencer.

69. The method of claim 24, further comprising preparing a computer readable record comprising a plurality of entries, each entry comprising a first identifier which corresponds to a first polypeptide sequence and a second identifier which corresponds to a binding property of the first polypeptide sequence.

70. A method for identifying nucleotide sequences encoding interacting polypeptide sequences, the method comprising:
providing a cell population comprising 100,000 bacterial host cells, wherein each of the 100,000 bacterial host cells contains a reporter gene operably linked to a transcriptional regulatory sequence which includes a binding site for a DNA-binding domain;
introducing into each of the 100,000 bacterial host cells a first chimeric gene encoding a first fusion protein, wherein the first fusion protein comprises a first polypeptide sequence and a DNA-binding domain, wherein the first chimeric gene comprises a first nucleotide sequence which encodes the first polypeptide sequence, and wherein the first nucleotide sequence is different in the first chimeric gene introduced into each of the 100,000 bacterial host cells;
introducing into each of the 100,000 bacterial host cells a second chimeric gene encoding a second fusion protein, wherein the second fusion protein comprises a second polypeptide sequence and an activation tag, wherein the second chimeric gene comprises a second nucleotide sequence which encodes the second polypeptide sequence, and wherein the second nucleotide sequence is different in the second chimeric gene introduced into each of the 100,000 bacterial host cells;
culturing the 100,000 bacterial host cells for a time sufficient to allow an interaction of the first fusion protein and the second fusion protein, if present, wherein the interaction results in a measurable change in expression of the reporter gene;
selecting from the 100,000 bacterial host cells those cells that exhibit the measurable change in expression of the reporter gene, to thereby result in selected bacterial host cells; and
sequencing the first nucleotide sequence and the second nucleotide sequence contained in selected bacterial host cells, to thereby identify nucleotide sequences encoding interacting polypeptide sequences.

71. A method for identifying nucleotide sequences encoding interacting polypeptide sequences, the method comprising:
providing a cell population comprising a plurality of host cells, wherein each of the plurality of host cells contains a reporter gene operably linked to a transcriptional regulatory sequence which includes a binding site for a DNA-binding domain;
introducing into each of the plurality of host cells a first chimeric gene encoding a first fusion protein, wherein the first fusion protein comprises a first polypeptide sequence and a DNA binding domain, and wherein the first chimeric gene comprises a first nucleotide sequence encoding the first polypeptide sequence;
introducing into each of the plurality of host cells a second chimeric gene encoding a second fusion protein, wherein the second fusion protein comprises a second polypeptide sequence and an activation tag, wherein the second chimeric gene comprises a second nucleotide sequence which encodes the second polypeptide sequence, and wherein the second nucleotide sequence is different in the second chimeric gene introduced into each of the plurality of host cells;
culturing the plurality of host cells for a time sufficient to allow an interaction of the first fusion protein and the second fusion protein, if present, wherein the interaction results in a measurable change in expression of the reporter gene;
selecting from the plurality of host cells those cells that exhibit the measurable change in expression of the reporter gene, to thereby result in selected host cells;
purifying the second nucleotide sequence from each of the selected host cells, wherein the purification is carried out by an automated process in compartments of a multi-compartment device; and
sequencing the second nucleotide sequence from each of the selected host cells, to thereby identify nucleotide sequences encoding interacting polypeptide sequences.

72. The method of claim 71, wherein the second nucleotide sequences from each of the selected host cells are purified in wells of a 96 well vessel.

73. The method of claim 72, wherein sequencing reactions for the second nucleotide sequences are carried out in wells of a 384 well vessel.

74. A method for identifying nucleotide sequences encoding interacting polypeptide sequences, the method comprising:
providing a cell population comprising a plurality of host cells, wherein each of the plurality of host cells contains a reporter gene operably linked to a transcriptional regulatory sequence which includes a binding site for a DNA-binding domain;
introducing into each of the plurality of host cells a first chimeric gene encoding a first fusion protein, wherein the first fusion protein comprises a first polypeptide sequence and a DNA binding domain, wherein the first chimeric gene comprises a first nucleotide sequence which encodes the first polypeptide sequence, and wherein the first nucleotide sequence is different in the first chimeric gene introduced into each of the plurality of host cells;
introducing into each of the plurality of host cells a second chimeric gene encoding a second fusion protein, wherein the second fusion protein comprises a second polypeptide sequence and an activation tag, and wherein the second chimeric gene comprises a second nucleotide sequence encoding the second polypeptide sequence;
culturing the plurality of host cells for a time sufficient to allow an interaction of the first fusion protein and the second fusion protein, if present, wherein the interaction results in a measurable change in expression of the reporter gene;
selecting from the plurality of host cells those cells that exhibit the measurable change in expression of the reporter gene, to thereby result in selected host cells;
purifying the first nucleotide sequence from each of the selected host cells, wherein the purification is carried out by an automated process in compartments of a multi-compartment device; and
sequencing the first nucleotide sequence from each of the selected host cells, to thereby identify nucleotide sequences encoding interacting polypeptide sequences.

75. The method of claim 74, wherein the first nucleotide sequences from each of the selected host cells are purified in wells of a 96 well vessel.

76. The method of claim 75, wherein sequencing reactions for the first nucleotide sequences are carried out in wells of a 384 well vessel.

77. The method of claim 1, further comprising shotgun sequencing a nucleic acid library or the genome of an organism or a virus, wherein the method identifies in serial or in parallel via a two-hybrid assay the protein-protein interactions of polypeptides encoded by the nucleic acid library or the genome of the organism or virus.

78. The method of claim 77, wherein nucleic acid vectors used to carry out the shotgun sequencing are the same as the nucleic acid vectors used to carry out the two-hybrid assay.

79. The method of claim 77, wherein the method identifies in serial the protein-protein interactions of polypeptides encoded by the nucleic acid library or the genome of the organism or virus.

80. The method of claim 77, wherein the method identifies in parallel the protein-protein interactions of polypeptides encoded by the nucleic acid library or the genome of the organism or virus.

81. The method of claim 1, wherein the selected host cell is grown, prior to sequencing of the first and second nucleotide sequences, in a well of a plate having at least 96 wells.

82. The method of claim 71, wherein each of the selected host cells are grown, prior to sequencing of the second nucleotide sequence from each of the selected host cells, in wells of plates having at least 96 wells.

83. The method of claim 74, wherein each of the selected host cells are grown, prior to sequencing of the first nucleotide sequence from each of the selected host cells, in wells of plates having at least 96 wells.
